(19) United States
(12) Patent Application Publication
(10) Pub. No.: US 2007/0129310 A1
(43) Pub. Date: Jun. 7, 2007

(54) Antibacterial agents

(75) Inventors: Raymond Paul Beckett, Terrytown, NY (US); Jac Wijkmans, Oss (NL); Thomas Krulle, Oxford (GB)

Correspondence Address: BANNER & WITCOFF, LTD., 1100 13th STREET, N.W., SUITE 1200, WASHINGTON, DC, 20005-4051, US

(73) Assignee: VERNALIS (OXFORD) LTD., WINNERSH, BERKSHIRE (GB)

(21) Appl. No.: 10/530,406

(22) PCT Filed: Dec. 2, 2002
(86) PCT No.: PCT/GB02/05409

§ 371 Date: Feb. 8, 2007

(30) Foreign Application Priority Data
Oct. 9, 2002 (GB) 0223532.3

(51) Int. Cl. A61K 38/05; A61K 38/04; A61K 31/541; A61K 31/5377; A61K 31/496; A61K 31/551; A61K 31/55; A61K 31/454; A61K 31/427

(52) U.S. Cl. 514/18; 514/19; 514/227.5; 514/237.8; 514/255.01; 514/247; 514/317; 514/217.12; 514/218; 514/365; 530/331; 544/60; 544/129; 544/162; 544/360; 544/386; 544/224; 544/238; 546/194; 548/200

(57) ABSTRACT

Compounds of formula (II) have antibacterial activity: wherein: Q represents a radical of formula —N(OH)CH(═0) or formula —C(═O)NH(OH); R¹ represents hydrogen, methyl or trifluoromethyl or, except when Q is a radical of formula —N(OH)CH(═0), a hydroxy, halo or amino group; R₂ represents a group R₁₀—(D)ₙ—(ALK)ₘ— wherein R₁₀ represents hydrogen or an optionally substituted C_{-C}₆ alkyl, C₂-C₆ alkynyl, C₂-C₆ alkynyl, cycloalkyl, aryl, or heterocyclyl group and ALK represents a straight or branched divalent C_{-C}₆ alkylene, C₂-C₆ alkynylene, or C₂-C₆ alkynylene radical, and may be interrupted by one or more non-adjacent —NH—, —O—or -Slinkages, D represents —NH—, —O—or —S—, and m and n are independently 0 or 1; R₄ represents the side chain of a natural or non-natural alpha amino acid; ring A represents an optionally substituted monocyclic heterocyclic ring containing from 5 to 7 ring atoms, one of which is the nitrogen atom shown, the remaining ring atoms being selected from compatible combinations of carbon, oxygen, sulfur and nitrogen; X is oxygen or sulfur; Y is oxygen, sulfur or —NH—; R is 0, 1, 2 or 3; and ring B represents an optionally substituted carbocyclic or heterocyclic ring system.

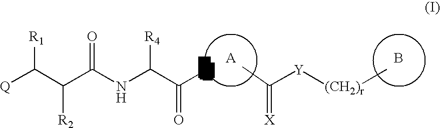

This invention relates to novel hydroxamic acid and N-formyl hydroxylamine derivatives having antimicrobial, particularly antibacterial activity, to methods of treatment using such compounds, and to pharmaceutical and veterinary compositions comprising such compounds.

### BACKGROUND TO THE INVENTION

Our copending International Patent Application No. WO 99/39704 describes and claims, inter alia, the use of N-formylhydroxylamine derivatives of formula (A) or a pharmaceutically or veterinarily acceptable salt thereof in the preparation of an antibacterial composition: 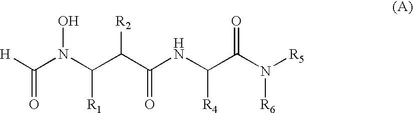
wherein R₁ represents hydrogen, C₁-C₆ alkyl or C₁-C₆ alkyl substituted by one or more halogen atoms; R₂ represents a substituted or unsubstituted C₁-C₆ alkyl, cycloalkyl(C₁-C₆ alkyl)- or aryl(C₁-C₆ alkyl)- group; R₄ represents the side chain of a natural or non-natural alpha amino acid, and R₅ and R₆ when taken together with the nitrogen atom to which they are attached form an optionally substituted saturated heterocyclic ring of 3 to 8 atoms which ring is optionally fused to a carbocyclic or second heterocyclic ring.

In addition our International Patent Application No. WO 99/59568 describes the use of analogues of the N-formylhydroxylamine derivatives of WO 99/39704 (wherein the N-formylhydroxylamine group is replaced by a hydroxamic acid group) in the preparation of an antibacterial composition.

Further, our international patent Application No. WO 01/10834 relates to a group of antibacterially active hydroxamic acid and N-formyl hydroxylamine compounds which differ in structure from those of International Patent Applications Nos. WO 99/59568 and WO 99/39704, principally in the nature of the —NR₅R₆ group.

### BRIEF DESCRIPTION OF THE INVENTION

This invention is based on the finding that certain hydroxamic acid and N-formyl hydroxylamine derivatives have antimicrobial activity, particularly antibacterial, and antifungal activity, and makes available a new group of such agents. It has been found that the compounds with which this invention is concerned are antibacterial with respect to a range of bacteria, with potency against Gram-positive organisms generally being greater than against Gram-negatives. Many of the compounds of the invention show activity against bacteria responsible for respiratory infections, such as Streptococcus pneumoniae and Haemophilus influenzae.

It is presently believed that their antibacterial activity is due, at least in part, to intracellular inhibition of bacterial polypeptide deformylase (PDF; EC 3.5.1.31).

The compounds with which the present invention is concerned differ from those of WO 99/59568, WO 99/39704 and WO 01/10834 principally in the nature of the group corresponding to —NR₅R₆ of formula (A). The structural differences present in the compounds of this invention can confer benefits in antimicrobial spectrum and potency relative to those of the three cited prior art applications.

### DETAILED DESCRIPTION OF THE INVENTION

The present invention provides a compound of formula (I), or a pharmaceutically or veterinarily acceptable salt, hydrate or solvate thereof 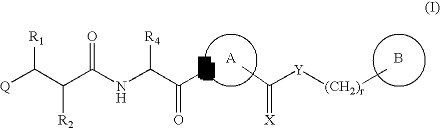
wherein:
Q represents a radical of formula —N(OH)CH(═O) or formula —C(═O)NH(OH);
R₁ represents hydrogen, methyl or trifluoromethyl or, except when Q is a radical of formula —N(OH)CH(═O), a hydroxy, halo or amino group;
R₂ represents a group R₁₀—(D)ₙ—(ALK)ₘ—wherein
   R₁₀ represents hydrogen, or an optionally substituted C₁-C₆ alkyl, C₂-C₆ alkenyl, C₂-C₆ alkynyl, cycloalkyl, aryl, or heterocyclyl group and
   ALK represents a straight or branched divalent C₁-C₆ alkylene, C₂-C₆ alkenylene, or C₂-C₆ alkynylene radical, and may be interrupted by one or more non-adjacent —NH—, —O— or —S— linkages,
   D represents —NH—, —O— or —S—, and
   m and n are independently 0 or 1;
R₄ represents the side chain of a natural or non-natural alpha amino acid;
   ring A represents an optionally substituted monocyclic heterocyclic ring containing from 5 to 7 ring atoms, one of which is the nitrogen atom shown, the remaining ring atoms being selected from compatible combinations of carbon, oxygen, sulfur and nitrogen;
X is oxygen or sulfur;
Y is oxygen, sulfur or —NH—;
R is 0, 1, 2 or3; and
   ring B represents an optionally substituted carbocyclic or heterocyclic ring system.

In another aspect, the invention provides a method for the treatment of microbial infections in humans and non-human mammals, which comprises administering to a subject suffering such infection an antimicrobially effective dose of a compound of formula (I) as defined above.

In a further aspect of the invention there is provided a method for the treatment of microbial contamination by applying an antimicrobially effective amount of a compound of formula (I) as defined above to the site of contamination.

The compounds of formula (I) as defined above may be used as component(s) of antimicrobial cleaning or disinfecting materials.

As used herein, “microbe” means a bacterial, fungal or protozoal microorganism.

As used herein the term “(C₁-C₆)alkyl” means a straight or branched chain alkyl moiety having from 1 to 6 carbon atoms, including for example, methyl, ethyl, n-propyl, isopropyl, n-butyl, isobutyl, sec-butyl, t-butyl, n-pentyl and n-hexyl.

As used herein the term “(C₂-C₆)alkenyl” means a straight or branched chain alkenyl moiety having from 2 to 6 carbon atoms having at least one double bond of either E or Z stereochemistry where applicable. The term includes, for example, vinyl, allyl, 1- and 2-butenyl and 2-methyl-2-propenyl.

As used herein the term “C₂-C₆ alkynyl” refers to straight chain or branched chain hydrocarbon groups having from two to six carbon atoms and having in addition one triple bond. This term would include for example, ethynyl, 1-propynyl, 1- and 2-butynyl, 2-methyl-2-propynyl, 2-pentynyl, 3-pentynyl, 4-pentynyl, 2-hexynyl, 3-hexynyl, 4-hexynyl and 5-hexynyl.

As used herein the term “cycloalkyl” means a saturated alicyclic moiety having from 3-8 carbon atoms and includes, for example, cyclopropyl, cyclobutyl, cyclopentyl, cyclohexyl, cycloheptyl and cyclooctyl.

As used herein the term “carbocyclic ring system” means a mono-, bi- or tri-cyclic radical containing only carbon atoms in the ring(s), and includes, for example phenyl, naphthyl, fluorenyl and phenanthryl.

As used herein the term “heterocyclic ring system” means a mono-, bi- or tri-cyclic radical containing at least one oxygen, sulfur or nitogen atom in the ring(s), and includes for example, ring systems wherein one of the rings is a heterocyclic ring as defined below, and the benzodioxolyl ring system.

As used herein the term “aryl” refers to a mono-, bi- or tri-cyclic carbocyclic aromatic group, and to groups consisting of two covalently linked monocyclic carbocyclic aromatic groups. Illustrative of such groups are phenyl, biphenyl and napthyl.

As used herein the term “heteroaryl” refers to a 5- or 6- membered aromatic ring containing one or more heteroatoms;. Illustrative of such groups are thienyl, furyl, pyrrolyl, imidazolyl, benzimidazolyl, thiazolyl, pyrazolyl, isoxazolyl, isothiazolyl, triazolyl, thiadiazolyl, oxadiazolyl, pyridinyl, pyridazinyl, pyrimidinyl, pyrazinyl, triazinyl.

As used herein the unqualified term “heterocyclyl” or “heterocyclic” includes “heteroaryl” as defined above, and in particular means a 5-7 membered aromatic or non-aromatic heterocyclic ring containing one or more heteroatoms selected from S, N and O, including for example, pyrrolyi, furanyl, thienyl, piperidinyl, imidazolyl, oxazolyl, isoxazolyl, thiazolyl, thiadiazolyl, pyrazolyl, pyridinyl, pyrrolidinyl, pyrimidinyl, morpholinyl, piperazinyl, indolyl, morpholinyl, benzofuranyl, pyranyl, isoxazolyl, benzimidazolyl, methylenedioxyphenyl, maleimido and succinimido groups.

Unless otherwise specified in the context in which it occurs, the term “substituted” as applied to any moiety herein means substituted with up to four substituents, each of which independently may be (C₁-C₆)alkyl, (C₁-C₆)alkoxy, hydroxy, mercapto, (C₁-C₆)alkylthio, amino, halo (including fluoro, chloro, bromo and iodo), cyano, trifluoromethyl, nitro, —COOH, —CONH₂, —COR^{A}, —COOR^{A}, —NHCOR^{A}, —CONHR^{A}, —NHR^{A}, —NR^{A}R^{B}, or —CONR^{A}R^{B} wherein R^{A} and R^{B} are independently a (C₁-C₆)alkyl group

As used herein the terms “side chain of a natural alpha-amino acid” and “side chain of a non-natural alpha-amino acid” mean the group R^{x} in respectively a natural and non-natural amino acid of formula NH₂—CH(R^{x})—COOH.

Examples of side chains of natural alpha amino acids include those of alanine, arginine, asparagine, aspartic acid, cysteine, cystine, glutamic acid, histidine, 5-hydroxylysine, 4-hydroxyproline, isoleucine, leucine, lysine, methionine, phenylalanine, proline, serine, threonine, tryptophan, tyrosine, valine, α-aminoadipic acid, a-amino-n-butyric acid, 3,4-dihydroxyphenylalanine, homoserine, α-methylserine, ornithine, pipecolic acid, and thyroxine.

In natural alpha-amino acid side chains which contain functional substituents, for example amino, carboxyl, hydroxy, mercapto, guanidyl, imidazolyl, or indolyl groups as in arginine, lysine, glutamic acid, aspartic acid, tryptophan, histidine, serine, threonine, tyrosine, and cysteine, such functional substituents may optionally be protected.

Likewise, in the side chains of non-natural alpha amino acids which contain functional substituents, for example amino, carboxyl, hydroxy, mercapto, guanidyl, imidazolyl, or indolyl groups, such functional substituents may optionally be protected.

The term “protected” when used in relation to a functional substituent in a side chain of a natural or non-natural alpha-amino acid means a derivative of such a substituent which is substantially non-functional. The widely used handbook by T. W. Greene and P. G. Wuts “Protective Groups in Organic Synthesis” Second Edition, Wiley, N.Y., 1991 reviews the subject. For example, carboxyl groups may be esterified (for example as a C₁-C₆ alkyl ester), amino groups may be converted to amides (for example as a NHCOC₁-C₆ alkyl amide) or carbamates (for example as an NHC(═O)OC₁-C₆ alkyl or NHC(═O)OCH₂Ph carbamate), hydroxyl groups may be converted to ethers (for example an OC₁-C6 alkyl or a O(C₁-C₆ alkyl)phenyl ether) or esters (for example a OC(═O)C₁-C₆ alkyl ester) and thiol groups may be converted to thioethers (for example a tert-butyl or benzyl thioether) or thioesters (for example a SC(═O)C₁-C₆ alkyl thioester).

There are several actual or potential chiral centres in the compounds according to the invention because of the presence of asymmetric carbon atoms. The presence of several asymmetric carbon atoms gives rise to a number of diastereoisomers with R or S stereochemistry at each chiral centre. The invention includes all such diastereoisomers and mixtures thereof. Currently, the preferred stereoconfiguration of the carbon atom carrying the R₂ group is R; that of the carbon atom carrying the R₄ group (when asymmetric) is S; and that of the carbon atom carrying the R₁ group (when asymmetric) is R.

In the compounds of the invention:

When Z is a radical of formula —N(OH)CH(═O), R₁ is hydrogen, methyl or trifluoromethyl. When Z is a radical of formula —C(═O)NH(OH), R₁ is hydrogen, methyl, trifluoromethyl, hydroxy, halo (e.g. chloro, bromo or especially fluoro) or amino. Hydrogen is currently preferred in both cases.

R₂ may be, for example:
optionally substituted C₁-C₈ alkyl, C₃-C₆ alkenyl, C₃-C₆ alkynyl or cycloalkyl;
phenyl(C₁-C₆ alkyl)-, phenyl(C₃-C₆ alkenyl)- or phenyl(C₃-C₆ alkynyl)- optionally substituted in the phenyl ring;
cycloalkyl(C₁-C₆ alkyl)-, cycloalkyl(C₃-C6 alkenyl)- or cycloalkyl(C₃-C₆ alkynyl)- optionally substituted in the cycloalkyl ring;
heterocyclyl(C₁-C₆ alkyl)-, heterocyclyl(C₃-C₆ alkenyl)- or heterocyclyl(C₃-C₆ alkynyl)- optionally substituted in the heterocyclyl ring; or
CH₃(CH₂)ₚO(CH₂)_{q}—or CH₃(CH₂)ₚS(CH₂)_{q}—, wherein p is 0, 1, 2, or 3 and q is 1, 2 or 3.

Specific examples of R₂ groups include
methyl, ethyl, n- and iso-propyl, n- and iso-butyl, n-pentyl, iso-pentyl 3-methyl-but-1-yl, n-hexyl, n-heptyl, n-acetyl, n-octyl, methylsulfanylethyl, ethylsulfanylmethyl, 2-methoxyethyl, 2-ethoxyethyl, 2-ethoxymethyl, 3-hydroxypropyl, allyl, 3-phenylprop-3-en-1-yl, prop-2-yn-1-yl, 3-phenylprop-2-yn-1-yl, 3-(2-chlorophenyl)prop-2-yn-1-yl, but-2-yn-1-yl, cyclopentyl, cyclohexyl, cyclopentylmethyl, cyclopentylethyl, cyclopentylpropyl, cyclohexylmethyl, cyclohexylethyl, cyclohexylpropyl, furan-2-ylmethyl, furan-3-methyl, tetrahydrofuran-2-ylmethyl, tetrahydrofuran-2-ylmethyl, piperidinylmethyl, phenylpropyl, 4-chlorophenylpropyl, 4-methylphenylpropyl, 4-methoxyphenylpropyl, benzyl, 4-chlorobenzyl, 4-methylbenzyl, and 4-methoxybenzyl.
Presently preferred groups at R₂ are (C₁-C₆)alkyl-, cycloalkylmethyl-, (C₁-C₃)alkyl-S-(C₁-C₃)alkyl-, or (C₁-C₃)alkyl-O-(C₁-C₃)alkyl-, especially n-propyl, n-butyl, n-pentyl, cyclopentylmethyl, cyclopentylethyl, cyclohexylmethyl or cyclohexylethyl.

R₄ may be, for example
the characterising group of a natural a amino acid, for example benzyl, or 4-methoxyphenylmethyl, in which any functional group may be protected, any amino group may be acylated and any carboxyl group present may be amidated; or
a group —[Alk]ₙR₉ where Alk is a (C₁—C₆)alkylene or (C₂—C₆)alkenylene group optionally interrupted by one or more —O—, or —S— atoms or —N(R₁₂)— groups [where R₁₂ is a hydrogen atom or a (C₁—C₆)alkyl group], n is 0 or 1, and R₉ is hydrogen or an optionally substituted phenyl, aryl, heterocyclyl, cycloalkyl or cycloalkenyl group or (only when n is 1) R₉ may additionally be hydroxy, mercapto, (C₁-C₆)alkylthio, amino, halo, trifluoromethyl, nitro, —COOH, —CONH₂, —COOR^{A}, —NHCOR^{A}, —CONHR^{A}, —NHR^{A}, —NR^{A}R^{B}, or —CONR^{A}R^{B} wherein R^{A} and R^{B} are independently a (C₁-C₆)alkyl group; or
a benzyl group substituted in the phenyl ring by a group of formula —OCH₂COR₈ where R₈ is hydroxyl, amino, (C₁-C₆)alkoxy, phenyl(C₁-C₆)alkoxy, (C₁-C6)alkylamino, di((C₁-C₆)alkyl)amino, phenyl(C₁-C₆)alkylamino; or
a heterocyclic(C₁-C₆)alkyl group, either being unsubstituted or mono- or di-substituted in the heterocyclic ring with halo, nitro, carboxy, (C₁-C₆)alkoxy, cyano, (C₁-C₆)alkanoyl, trifluoromethyl (C₁-C₆)alkyl, hydroxy, formyl, amino, (C₁-C₆)alkylamino, di-(C₁-C₆)alkylamino, mercapto, (C₁-C₆)alkylthio, hydroxy(C₁-C6)alkyl, mercapto(C₁-C₆)alkyl or (C₁-C₆)alkylphenylmethyl; or
a group —CRₐR_{b}R_{c} in which:
   each of Rₐ, R_{b} and R_{c} is independently hydrogen, (C₁-C₆)alkyl, (C₂-C₆)alkenyl, (C₂-C₆)alkynyl, phenyl(C₁-C₆)alkyl, (C₃-C₈)cycloalkyl; or
   R_{c} is hydrogen and Rₐ and R_{b} are independently phenyl or heteroaryl such as pyridyl; or
   R_{c} is hydrogen, (C₁-C₆)alkyl, (C₂-C₆)alkenyl, (C₂-C₆)alkynyl, phenyl(C₁-C₆)alkyl, or (C₃-C₈)cycloalkyl, and Rₐ and R_{b} together with the carbon atom to which they are attached form a 3 to 8 membered cycloalkyl or a 5- to 6-membered heterocyclic ring; or
   Rₐ, R_{b} and R_{c} together with the carbon atom to which they are attached form a tricyclic ring (for example adamantyl); or
   Rₐ and R_{b} are each independently (C₁-C₆)alkyl, (C₂-C₆)alkenyl, (C₂-C₆)alkynyl, phenyl(C₁-C₆)alkyl, or a group as defined for R_{c} below other than hydrogen, or Rₐ and R_{b} together with the carbon atom to which they are attached form a cycloalkyl or heterocyclic ring, and R_{c} is hydrogen, —OH, —SH, halogen, —CN, —CO₂H, (C₁-C₄)perfluoroalkyl, —CH₂OH, —CO₂(C₁-C₆)alkyl, —O(C₁-C₆)alkyl, —O(C₂-C₆)alkenyl, —S(C₁-C₆)alkyl, —SO(C₁-C₆)alkyl, —SO₂(C₁-C₆) alkyl, —S(C₂-C₆)alkenyl, —SO(C₂-C₆)alkenyl, —SO₂(C₂-C₆)alkenyl or a group —Q—W wherein Q represents a bond or —O—, —S—, —SO— or —SO₂— and W represents a phenyl, phenylalkyl, (C₃-C₈)cycloalkyl, (C₃-C₈)cycloalkylalkyl, (C₄-C₈)cycloalkenyl, (C₄-C₈)cycloalkenylalkyl, heteroaryl or heteroarylalkyl group, which group W may optionally be substituted by one or more substituents independently selected from, hydroxyl, halogen, —CN, —CO₂H, —CO₂(C₁-C₆)alkyl, —CONH₂, —CONH(C₁-C₆)alkyl, —CONH(C₁-C₆alkyl)₂, —CHO, —CH₂OH, (C₁-C₄)perfluoroalkyl, —O(C₁-C₆)alkyl, —S(C₁-C₆)alkyl, —SO(C₁-C₆)alkyl, —SO₂(C₁-C₆)alkyl, —NO₂, —NH₂, —NH(C₁-C₆)alkyl, —N((C₁ -C₆)alkyl)₂, —NHCO(C₁-C₆)alkyl, (C₁-C₆)alkyl, (C₂-C₆)alkenyl, (C₂-C₆)alkynyl, (C₃-C₈)cycloalkyl, (C₄-C₈)cycloalkenyl, phenyl or benzyl.

Examples of particular R₄ groups include methyl, ethyl, benzyl, 4-chlorobenzyl, 4-hydroxybenzyl, phenyl, cyclohexyl, cyclohexylmethyl, pyridin-3-ylmethyl, tert-butoxymethyl, naphthylmethyl, iso-butyl, sec-butyl, tert-butyl, 1-benzylthio-1-methylethyl, 1-methylthio-1-methylethyl, 1-mercapto-1-methylethyl, 1-methoxy-1-methylethyl, 1-hydroxy-1-methylethyl, 1-fluoro-1-methylethyl, hydroxymethyl, 2-hydroxethyl, 2-carboxyethyl, 2-methylcarbamoylethyl, 2-carbamoylethyl, and 4-aminobutyl. Presently preferred R₄ groups include tert-butyl, iso-butyl, benzyl, isopropyl and methyl.

Examples of rings A are optionally substituted 1-pyrrolidinyl, piperidin-1-yl, 1-piperazinyl, hexahydro-1-pyridazinyl, morpholin-4-yl, tetrahydro-1,4-thiazin-4-yl, tetrahydro-1,4-thiazin-4-yl 1-oxide, tetrahydro-1,4-thiazin-4-yl 1,1-dioxide, hexahydroazipino, thiomorpholino, diazepino, thiazolidinyl or octahydroazocino. Presently preferred rings A are piperidin-1-yl and 1-piperazin-4-yl. The grouping 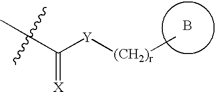
present in compounds (I) may be attached to a ring carbon atom or a second ring nitrogen atom of ring A.

At present it is preferred that r is 0 or 1.

Examples of rings B are optionally substituted phenyl, 2-, 3- or 4-pyridyl, 9H-fluoren-9-yl, naphthyl, and 4-benzo[1,3]dioxol-5-yl.

In the grouping 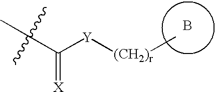
present in compounds (I), it is presently preferred that X is oxygen or sulphur when Y is —NH—, or both X and Y are oxygen, and that r is 0 or 1

Examples of specific compounds of the invention are those of the Examples herein. In those Examples, where a compound of formula (I) above wherein Q is an N-formylhydroxylamine radical —N(OH)CH(═O) is disclosed, it is to be understood that the equivalent compound wherein Q is a hydroxamate radical —C(═O)NH(OH) is also a specific compound of the invention, and vice versa.

Preferred compounds of the invention include those selected from the group consisting of compounds of formulae (IID) - (IIG) and (IIW) - (IIZ): 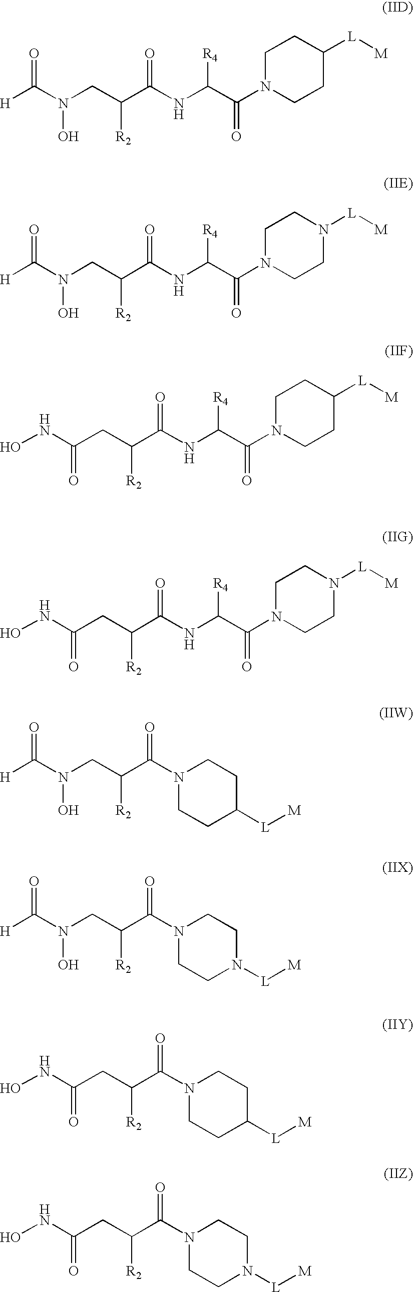
wherein
R₂ is n-propyl, n-butyl, n-pentyl, cyclopentylmethyl, cyclopentylethyl, cyclohexylmethyl or cyclohexylethyl;
R₄ is tert-butyl, iso-butyl, benzyl or methyl;
L is —C(═O)O—, —C(═O)NH— or —C(═S)NH— and
M is a phenyl, benzyl, naphthyl, 3,4-methylenedioxyphenyl (ie 4-benzo[1,3]dioxol-5-yl), or 9H-fluoren-9-ylmethyl group, which may optionally be substituted, for example by (C₁-C₃)alkyl, (C₁-C₃)alkoxy, hydroxy, mercapto, (C₁-C₃)alkylthio, amino, halo (eg chloro), cyano, trifluoromethyl, nitro, —COOH, —CONH₂, —COR^{A}, —COOR^{A}, —NHCOR^{A}, —CONHR^{A}, —NHR^{A}, —NR^{A}R^{B}, or —CONR^{A}R^{B} wherein R^{A} and R^{B} are independently a (C₁-C₃)alkyl group.

Compounds of the invention in which Q is an N-formylhydroxyamino group may be prepared by deprotecting an O-protected N-formyl-N-hydroxyamino 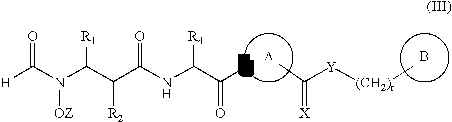
compound of formula (III):
in which R₁, R₂, R4, X, Y, r and rings A and B are as defined for general formula (I) and Z is a hydroxy protecting group removable to leave a hydroxy group by hydrogenolysis or hydrolysis. Benzyl is a preferred Z group for removal by hydrogenolysis, and tert-butyl and tetrahydropyranyl are preferred groups for removal by acid hydrolysis.

Compounds of the invention in which Q is a hydroxamic acid group may be prepared by reacting the parent compound wherein Q is a carboxylic acid 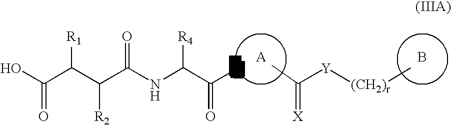
group (IIIA)
with hydroxylamine or an N- and/or O-protected hydroxylamine, and thereafter removing any O- or N-protecting groups

Compounds of formula (III) or (IIIA) may be prepared by causing an acid of formula (IV) or (IVA) 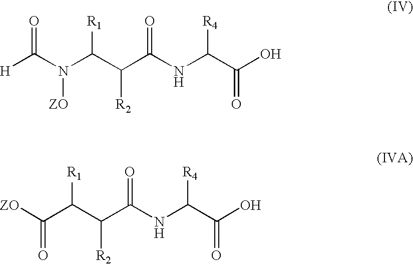
or an activated derivative thereof to react with an amine of formula (V)
wherein R₁, R₂, R4, X, Y, Z, r and rings A and B are as defined for general formula are as defined in general formula (II) and Z is as defined in relation to formula (III) above, then in the case of the reaction product of (IVA) and (V) removing the O-protecting group Z.

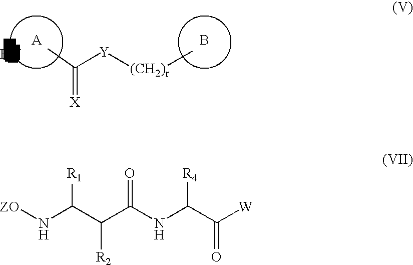

Compounds of formula (IV) may be prepared by N-formylation, for example using acetic anhydride and formic acid, or 1-formylbenzotriazole, of compounds of formula (V)

wherein R₁, R₂, R₄, and Z are as defined in relation to formula (III) and W is either a chiral auxiliary or an OZ¹ group wherein Z¹ is hydrogen or a hydroxy protecting group. In the case where W is an OZ¹ group or a chiral auxiliary the hydroxy protecting group or auxiliary is removed after the formylation step to provide the compound of formula (V). Suitable chiral auxiliaries include substituted oxazolidinones which may be removed by hydrolysis in the presence of base.

A compound of general formula (IVA) may be prepared by reduction of an oxime of general formula (VIII) 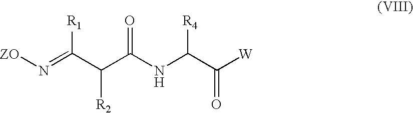
wherein R₁, R₂, R₄, and Z are as defined above, and W is either an OZ¹ group as defined above or a chiral auxiliary. Reducing agents include certain metal hydrides (eg sodium cyanoborohydride in acetic acid, triethylsilane or borane/pyridine) and hydrogen in the presence of a suitable catalyst. Following the reduction when the group W is a chiral auxiliary it may be optionally converted to a OZ¹ group.

A compound of general formula (VIII) can be prepared by reaction of a β-keto carbonyl compound of general formula (IX) 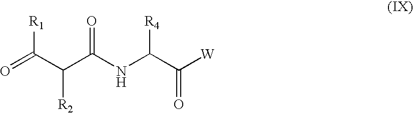
wherein R₁, R₂, R₄ and W are as defined above, with an O-protected hydroxylamine.

β-keto carbonyl compounds (IX) may be prepared in racemic form by formylation or acylation of a carbonyl compound of general formula (X) 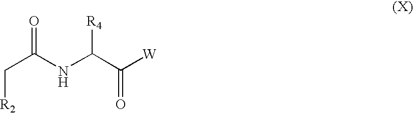
wherein R₂, R₄, and W are as defined above, with a compound of general formula (X)
R₁(C═O)Q¹
wherein R₁ is as defined above and Q¹ is a leaving group such as halogen or alkoxy, in the presence of a base.

A compound of general formula (V) may be prepared by reduction of an oxime of general formula (VII) 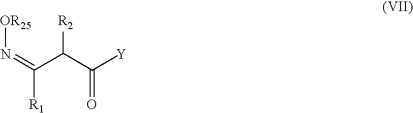
wherein R₁, R₂, and R₂₅ are as defined above, and Y is either an OR₂₆ group as defined above or a chiral auxiliary. Reducing agents include certain metal hydrides (eg sodium cyanoborohydride in acetic acid, triethylsilane or borane/pyridine) and hydrogen in the presence of a suitable catalyst. Following the reduction when the group Y is a chiral auxiliary it may be optionally converted to a OR₂₆ group.

Compounds of formula (V) may be prepared by standard literature methods, and by analogy with the methods and routes described in the Examples herein.

In the Examples, the following abbreviations have been used throughout:
DCM Dichloromethane
DIEA Diisopropylethylamine
DMF Dimethylformamide
HATU O-(7-Azabenzotriazol-1-yl)-1,1,3,3-tetramethyluronium hexafluorophosphate
HOBt 1-Hydroxy-7-benzotriazole
HPLC High performance liquid chromatography
LRMS Low resolution mass spectrometry
NMR Nuclear Magnetic Resonance
PyAOP 7-Azabenzotriazol-1-yl-oxy-tris-pyrrolidino-phosphonium hexafluorophosphate
rt Room temperature
RT Retention time
TBTU 2-(1H-Benzotriazole-1-yl)-1,1,3,3-tetramethyluronium tetrafluorophosphate
TFA Trifluoroacetic acid

¹H and ¹³C spectra were recorded using a Bruker DPX 250 spectrometer at 250.1 MHz (62.5 MHz for the ¹³C). Chemical shift values are expressed in δ (ppm) and abbreviations are as follows: s=singlet, d=doublet, t=triplet, q=quartet, dd=double doublet, m=multiplet, b=broad and app=apparent. Mass spectra were obtained using a Perkin Elmer Sciex API 165. Analytical HPLC was run on a Beckman System Gold, using Waters Symmetry C18 column (50 mm, 4.6 mm) with 20 to 90% solvent B gradient (1.5 mvmin) as the mobile phase. [Solvent A: 0.05% TFA in 10% MeCN 90% water, Solvent B: 0.05% TFA in 10% water 90% MeCN, 5 min gradient time], detection wavelength at 220 or 214 nm. Preparative HPLC was run on a Gilson autoprep instrument using a C18 Waters delta pak (15 μm, 300 Å, 25 mm, 100 mm) with 10 to 90% solvent B gradient as the mobile phase at a flow rate of 15 ml/min. [Solvent A 10% MeCN/water; Solvent B: 10% water/MeCN, 8 min gradient time], UV detection was at 220 or 214 nm.

General Schemes

N-mono-substituted Piperazines and diazepines are either commercially available or were synthesised by the following route:

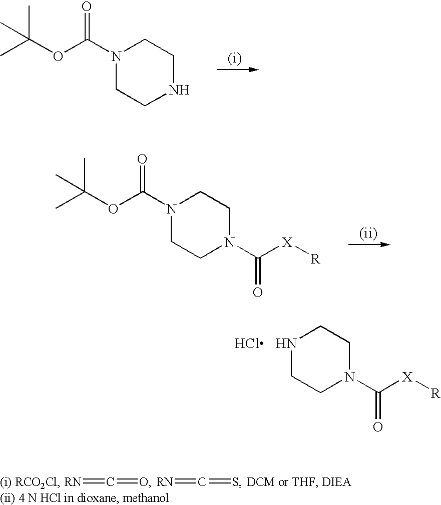

Extended acid components were synthesised by the following route and experimental data is given below.

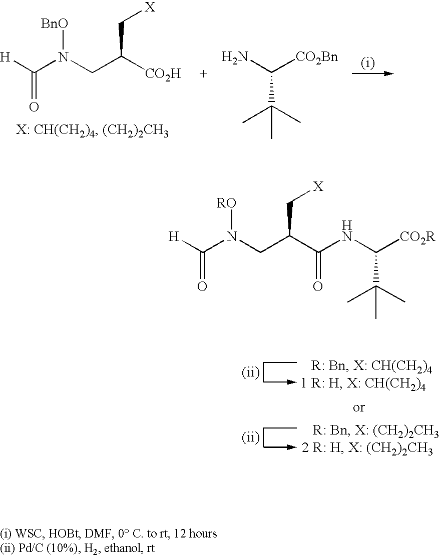

N-Formyl hydroxylamines were synthesised by the following route 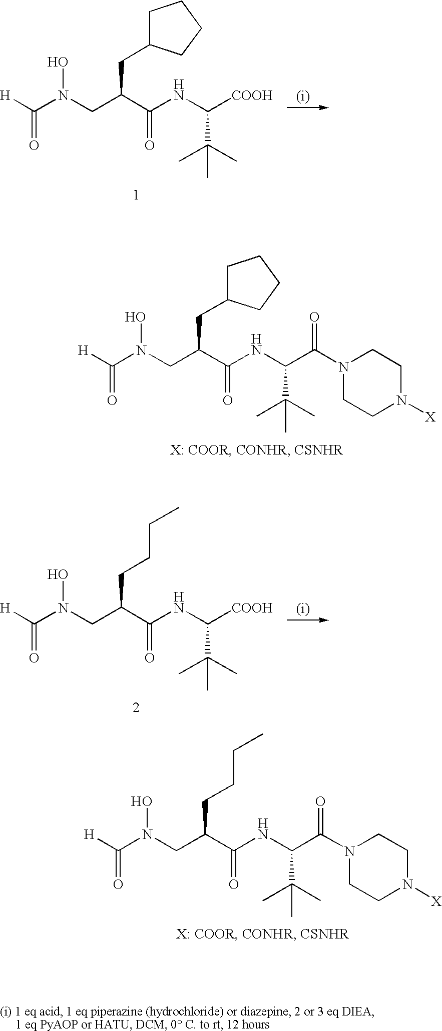
Preparation of Intermediate 1

### 2S-[3-(Benzyloxy-formyl-amino)-2R-cyclopentylmethyl-)-proplonylamino]-3,3-dimethyl-butyric acid benzyl ester

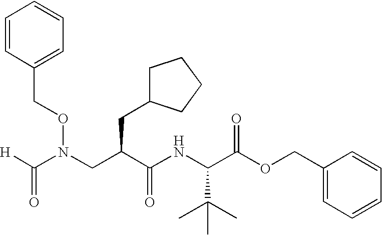

1-(3-Dimethylaminopropyl)-3-ethylcarbodiimide hydrochloride (11.0 g, 57.4 mmol), HOBt monohydrate (7.8 g, 50.9 mmol) and 2SAmino-3,3-dimethyl-butyric acid benzyl ester (13.8 g, 62.4 mmol) were added to a solution of 3-(Benzyloxy-formyl-amino)-2R-cyclopentylmethyl-propionic acid (14.7 g, 48.1 mmol), in dry DMF (150 ml) at 0° C. The ice bath was removed after 2.5 hours and the mixture was stirred for further 10 hours at room temperature. After removal of the solvent reduced pressure the oily residue was taken up in ethyl acetate (700 ml), washed with sat. sodium bicarbonate solution (150 ml) and brine (150 ml) before dried over anhydrous magnesium sulphate. Concentration and purification by silica gel flash chromatography (eluent: 8/1 toluene/acetone) gave a solid material, which was recrystallised from ethyl acetate/hexane to give the title compound (15.8 g, 65%) as off-white crystals. LRMS: +ve ion 509 [M+H⁺, 100%], −ve ion [M−H⁺, 507, 100%]

### 2S-[2R-Cyclopentylmethyl-3-(formyl-hydroxy-amino)-propionylamino]-3,3-dimethyl-butyric acid (Intermediate 1)

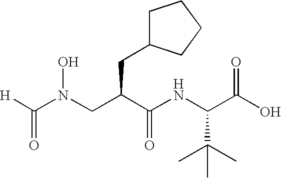

A mixture of 2S-[3-(Benzyloxy-formyl-amino)-2R-cyclopentylmethyl-)-propionylamino]-3,3-dimethyl-butyric acid benzyl ester (6.44 g, 12.7 mmol) and Palladium-on-carbon (10%, 560 mg) in ethanol (75 ml) was stirred under an atmosphere of hydrogen for 12 hours. Filtration over celite and concentration gave an oily residue, which was taken up in ethyl acetate (300 ml) and filtered by gravitation. Removal of the solvent under reduced pressure gave the title Intermediate 1 (3.91 g, 94%) as a pink foam. LRMS: +ve ion 329 [M+H⁺, 100%], −ve ion [M−H⁺, 327, 100%]; prep HPLC-RT: 8.8 min.

Preparation of Intermediate 2

### 2S-[2R-(Benzyloxy-formyl-amino)-methyl-)hexanoylamino}-3,3-dimethyl-butyric acid benzyl ester (Intermediate 2)

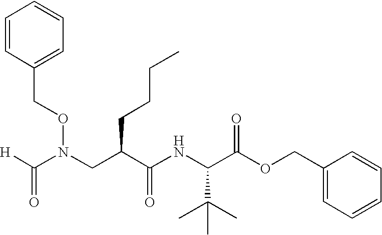

1-(3-Dimethylaminopropyl)-3-ethylcarbodiimide hydrochloride (1.20 g, 4.90 mmol), HOBt monohydrate (0.90 g, 5.88 mmol) and 2S-Amino-3,3-dimethyl-butyric acid benzyl ester (1.30 g, 5.87 mmol) were added to a solution of 2R-[(Benzyloxy-formyl-amino)-methyl]-hexanoic acid (1.37 9, 4.90 mmol) in dry DMF (20 ml) at 0° C. The ice bath was removed after 2 hours and the mixture was stirred for further 10 hours at room temperature. The mixture was taken up in ethyl acetate (250 ml), washed with citric acid solution (5%, 50 ml), sat. sodium bicarbonate solution (2×50 ml) and brine (50 ml) and dried over anhydrous magnesium sulphate. Concentration and purification by silica gel flash chromatography (eluent: 8/1 toluene/acetone) yielded the title compound (1.74 g, 74%) as an oil. LRMS: +ve ion 483 [M+H⁺, 100%]; ¹H-NMR (250 MHz), δ (CDCl₃) 8.12, 7.88 (1H, 2 bs, CHO-rotamers), 7.36-7.30 (10H, m, 10 ArH), 6.02 (1H, bd, J 9.0 Hz, NH), 5.14 (2H, AB-system, CO₂CH₂Ph), 4.96-4.69 (2H, m, NOCH₂Ph), 4.43 (1H, d, J 9.0 Hz, tert-ButylCH), 3.74, 3.10 (2H, 2 m, ONCH₂), 2.55 (1H, m, CH₂CHCH₂), 1.70-0.81 (9H, 2 m, CH(CH₂)₃, CH₃), 0.91 (9H, s, C(CH₃)₃).

### 2S-{2R-[(Formyl-hydroxy-amino)-methyl]-hexanoylamino}-3,3-dimethyl-butyric acid (Intermediate 2)

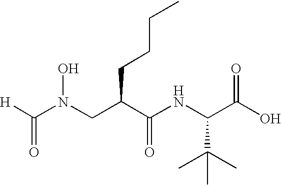

A mixture of 2S-[2R-(Benzyloxy-formyl-amino)-methyl-)-hexanoylamino}-3,3-dimethyl-butyric acid benzyl ester (1.74 g, 3.61 mmol) and Palladium-on-carbon (10%, 202 mg) in ethanol (50 ml) was stirred under an atmosphere of hydrogen for 2 hours. Filtration over celite and concentration gave an oily residue, which was taken up in ethyl acetate (200 ml) and filtered by gravitation. Removal of the solvent under reduced pressure gave the title Intermediate 2 (1.08 g, quant) as a pink foam. LRMS: +ve ion 325 [M+Na⁺, 100%], −ve ion [M−H⁺, 301, 100%]; ¹H-NMR (250 MHz), δ (MeOH-D4) 8.25, 7.82 (1H, 2 bs, CHO-rotamers), 4.31 (1H, m, tert-ButylCH), 3.78 (1H, dd, J₁ 9.5 Hz, J₂ 14.2 Hz, 0.5 HONCH₂), 3.42 (1H, dd, J₁ 4.7 Hz, J₂ 14.2 Hz, 0.5 HONCH₂), 3.04 (1H, m, CH₂CHCH₂), 1.57-0.90 (9H, 2 m, CH(CH₂)₃, CH₃), 1.03, 1.01 (9H, 2 s, C(CH₃)₃-rotamers).

### EXAMPLE 1

### 4-{2S-[2R-Cyclopentylmethyl-3-(formyl-hydroxy-amino)-propionylamino]-3,3-dimethyl-butyryl}-piperazine-1-carboxylic acid benzyl ester

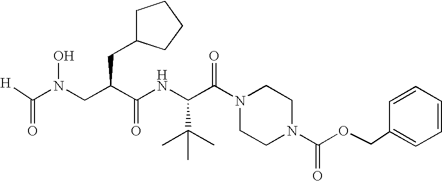

To a suspension of 2S-[2R-Cyclopentylmethyl-3-(formyl-hydroxy-amino)-propionylamino]-3,3-dimethyl-butyric acid Intermediate 1 (203 mg, 0.61 mmol) in dry DCM (15 ml) at 0° C. Piperazine-1-carboxylic acid benzyl ester hydrochloride (0.140 g, 0.64 mmol), 2,4,6-Collidine (0.25 ml, 1.89 mmol) and HATU (235 mg, 0.62 mmol) were added successively. The ice bath was removed after 1 hour and the reaction mixture was further stirred for 10 hours at room temperature. The reaction mixture was taken up in ethyl acetate (100 ml) washed with aqueous citric acid solution (5%, 20 ml), saturated sodium bicarbonate solution (20 ml), water (20 ml) and brine (20 ml) and dried over anhydrous magnesium sulphate. Concentration and purification by preparative HPLC gave the title compound (25 mg, 8%) as a colourless oil. LRMS: −ve ion 529 [M−H, 100%]; HPLC-RT: 10.9 min; ¹H-NMR (250 MHz), δ (MeOH-d4): 8.25, 7.81 (1H, 2 bs, CHO-rotamers), 7.37-7.30.(5H, m, ArH), 5.13 (2H, m, CH₂Ph), 4.90 (1H, m, tert-ButylCH), 3.91-2.71 (11H, 5m, 4 CH₂N-piperazine ,HONCH₂, CH₂CHCH₂), 1.89-0.99 (20H, 2 m, 4 CH₂-cyclopentyl, CH-cyclopentyl, CH₂, C(CH₃)₃).

### EXAMPLE 2

### 4-{2S-[2R-Cyclopentylmethyl-3-(formyl-hydroxy-amino)-propionylamino]-3,3-dimethyl-butyryl}-piperazine-1-carboxylic acid 9H-fluoren-9-ylmethyl ester

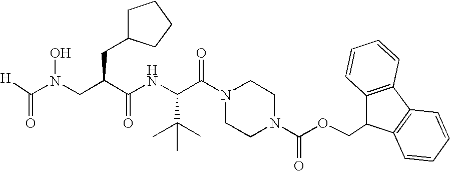

2S-[2R-Cyclopentylmethyl-3-(formyl-hydroxy-amino)-propionylamino]-3,3-dimethyl-butyric acid Intermediate 1 (200 mg, 0.61 mmol), Piperazine-1-carboxylic acid 9H-fluoren-9-yl methylester hydrochloride (220 mg, 0.64 mmol), 2,4,6-Collidine (0.25 ml, 1.89 mmol) and HATU (230 mg, 0.62 mmol) were reacted in dry DCM (15 ml) under the same conditions employed to synthesise Example 1. Similar work-up and purification by preparative HPLC yielded the title compound (220 mg, 58%) as a colourless oil. LRMS: +ve ion 619 [M+H⁺, 100%]; HPLC-RT: 12.7 min; ¹H-NMR (250 MHz), δ (MeOH-d4): 8.25, 7.81 (1H, 2 bs, CHO-rotamers), 7.79 (2H, d, J=7.9 Hz, 2 ArH), 7.60 (2H, d, J=7.3 Hz, 2 ArH), 7.42-7.28 (4H, m, 4 ArH), 4.86 (1H, m, tert-ButylCH), 4.56 (2H, m, OCH₂), 3.82-3.01 (11H, 3 m, 4 CH₂N-piperazine, HONCH₂, CH₂CHCH₂), 1.82-0.98 (20H, 2 m, 4 CH₂-cyclopentyl, CH-cyclopentyl, CH₂, C(CH₃)₃).

### EXAMPLE 3

### 4-{2S-[2R-Cyclopentylmethyl-3-(formyl-hdroxy-amino)-propionylamino]-3,3-dimethy-butyryl}-piperazine-1-carboxylic acid 4-chloro-phenyl ester

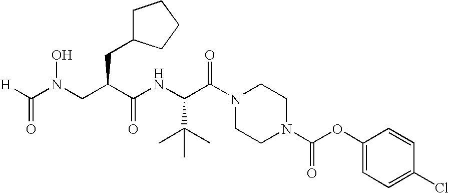

2S-[2R-Cyclopentylmethyl-3-(formyl-hydroxy-amino)-propionylamino]-3,3-dimethyl-butyric acid Intermediate 1 (150 mg, 0.46 mmol), Piperazine-1-carboxylic acid 4-chloro-phenylester hydrochloride (152 mg, 0.48 mmol), 2,4,6-Collidine (0.20 ml, 1.51 mmol) and HATU (175 mg, 0.46 mmol) were reacted in dry DCM (15 ml) under the same conditions employed to synthesise example 1. Similar work-up and purification by preparative HPLC yielded the title compound (0.210 g, 83%) as a colourless oil. LRMS: +ve ion 573 [M+Na⁺, 100%]; HPLC-RT: 11.3 min; ¹H-NMR (250 MHz), δ (MeOH-d4) 8.26, 7.82 (1H, 2 bs, CHO-rotamers), 7.41-7.35 (2H, m, ArH), 7.16-7.11 (2H, m, ArH), 4.92 (1H, m, tert-ButylCH), 3.99-3.06 (11H, 4 m, 4 CH₂N-piperazine, HONCH₂, CH₂CHCH₂), 2.03-1.02 (20H, 2 m, 4 CH₂-cyclopentyl, CH-cyclopentyl, CH₂, C(CH₃)₃).

### EXAMPLE 4

### 4-{2S-[2R-Cyclopentylmethyl-3-(formyl-hydroxy-amino)-propionylamino]-3,3-dimethyl-butyryl}-piperazine-1-carboxylic acid phenyl ester

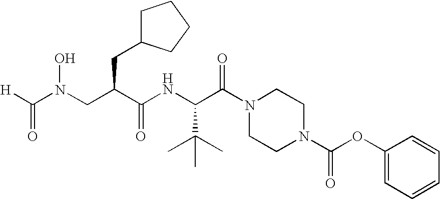

2S-[2R-Cyclopentylmethyl-3-(formyl-hydroxy-amino)-propionylamino]-3,3-dimethyl-butyric acid Intermediate 1 (2.0 g, 6.09 mmol), Piperazine-1-carboxylic acid 4-phenyl ester hydrochloride (1.48 g, 6.10 mmol), 2,4,6-Collidine (2.4 ml, 18.2 mmol) and HATU (2.3 g, 6.04 mmol) were reacted in dry DCM (50 ml) under the same conditions employed to synthesise example 1. Similar work-up (on a larger scale) and purification by preparative HPLC yielded the title compound (1.39 g, 44%) as a colourless foam. LRMS: +ve ion 539 [M+Na⁺, 100%]; HPLC-RT: 10.65 min; ¹H-NMR (250 MHz), δ (MeOH-d4) 8.26, 7.82 (1H, 2 bs, CHO-rotamers), 7.42-7.34 (2H, m, 2 ArH), 7.25-7.19 (1H, m, ArH), 7.13-7.09 (2H, m, 2 ArH), 4.93 (1H, m, tert-ButylCH), 4.00-2.91 (11H, 3 m, 4 CH₂N-piperazine, HONCH₂, CH₂CHCH₂), 1.89-1.02 (20H, 2 m, 4

### EXAMPLE 5

### 4-(2S-{2R-[(Formyl-hydroxy-amino)-methyl]-hexanoylamino}-3,3-dimethyl-butyryl)-piperazine-1-carboxylic acid benzyl ester

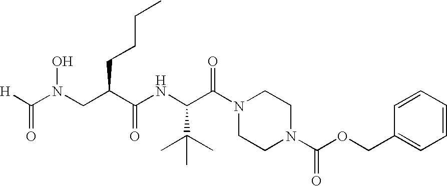

2S-{2R-[(Formyl-hydroxy-amino)-methyl]-hexanoylamino}-3,3-dimethyl-butyric acid Intermediate 2 (290 mg, 0.96 mmol), Piperazine-1-carboxylic acid benzyl ester hydrochloride (211 mg, 0.96 mmol), 2,4,6-Collidine (0.35 ml, 2.65 mmol) and HATU (365 mg, 0.96 mmol) were reacted in dry DCM (15 ml) under the same conditions employed to synthesise example 1. Similar work-up and purification by preparative HPLC yielded the title compound (93 mg, 19%) as a colourless oil. LRMS: +ve ion 527 [M+Na⁺, 50%]; HPLC-RT: 10.5 min; ¹H-NMR (250 MHz), δ (MeOH-d4) 8.25, 7.82 (1H, 2 bs, CHO-rotamers), 7.48, 7.28 (5H, m, 5 ArH), 5.14-5.12 (2H, m, OCH₂Ph), 4.88 (1H, bs, tert-ButylCH), 3.89-2.76 (11H, 4 m, 4 CH₂N-piperazine, HONCH₂, CH₂CHCH₂), 1.54-1.18 (6H, 2 m, CH(CH₂)₃), 1.11-0.99 (9H, s, C(CH₃)₃), 0.86 (3H, app.t, CH₃).

### EXAMPLE 6

### 4-{2S-[2R-Cyclopentylmethyl-3-(formyl-hydroxy-amino)-propionylamino]-3,3-dimethyl-butyryl}-[1,4]diazepane-1-carboxylic acid benzyl ester

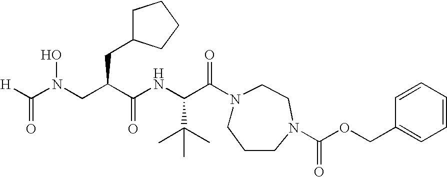

2S-[2R-Cyclopentylmethyl-3-(formyl-hydroxy-amino)-propionylamino]-3,3-dimethyl-butyric acid Intermediate 1 (200 mg, 0.62 mmol), Benzyl 1-homopiperazine-carboxylate (126 mg, 0.61 mmol), 2,4,6-Collidine (0.16 ml, 1.21 mmol) and PyAOP (316 mg, 0.61 mmol) were reacted in dry DCM (10 ml) under the same conditions employed to synthesise example 1. Similar work-up and purification by prep. HPLC yielded the title compound (83 mg, 25%) as a colourless oil. LRMS: +ve ion 545 [M+H⁺, 40%], 567 [M+Na⁺, 60%]; HPLC-.RT: 11.0 min; ¹H-NMR (250 MHz); δ (CDCl₃) 8.38, 7.78 (1H, 2 s, CHO-rotamers), 7.40-7.29 (5H, m, ArH), 6.74 (1H, m, NH), 5.13 (2H, AB-system, CH₂Ph), 4.84 (1H, m, tert-ButylCH), 4.16-2.73 (11H, m, 4 CH₂N-piperazine, HONCH₂, CH₂CHCH₂), 2.12-0.99 (13H, m, 4 CH₂-cyclopentyl, CH-cyclopentyl, CHCH₂CH, CH₂CH₂CH₂). 0.98, 0.95 (9H, 2 s, (CH₃)₃C-rotamers)

### EXAMPLE 7

### 4-{2S-[2R-Cyclopentylmethyl-3-(formyl-hydroxy-amino)-propionylamino]-3,3-dimethyl-butyryl)-piperazine-1-carboxylic acid (4-ethoxy-phenyl)-amide

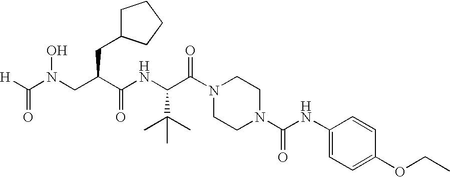

2S-[2R-Cyclopentylmethyl-3-(formyl-hydroxy-amino)-propionylamino]-3,3-dimethyl-butyric acid Intermediate 1 (134 mg, 0.41 mmol), Piperazine-1-carboxylic acid (4-ethoxy-phenyl)-amide hydrochloride (112 mg, 0.39 mmol), 2,4,6-Collidine (0.15 ml, 1.14 mmol) and HATU (150 mg, 0.39 mmol) were reacted in dry DCM (10 ml) under the same conditions employed to synthesise example 1. Similar work-up and purification by preparative HPLC yielded the title compound (70 mg, 32%) as a colourless oil. LRMS: +ve ion 560 [M+H⁺, 100%]; HPLC-RT: 9.3 min; ¹H-NMR (250 MHz), δ (MeOH-d4) 8.26, 7.82 (1H, 2 s, CHO-rotamers), 7.25-6.80 (4H, 2 m, ArH), 4.93 (1H, m, tert-ButylCH), 3.99 (2H, q, J 7.0 Hz, OCH₂CH₃), 3.92-2.81 (11H, 4 m, 4 CH₂N-piperazine, HONCH₂, CH₂CHCH₂), 1.89-1.01 (23H, 2 m, 4 CH₂-cyclopentyl, CH-cyclopentyl, CH₂, CH₃, C(CH₃)₃).

### EXAMPLE 8

### 4-{2S-[2R-Cyclopentylmethyl-3-(formyl-hydroxy-amino)-propionylamino]-3,3-dimethyl-butyryl}-piperazine-1-carboxylic acid naphthalen-2-ylamide

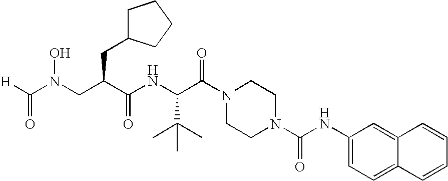

2S-[2R-Cyclopentylmethyl-3-(formyl-hydroxy-amino)-propionylamino]-3,3-dimethyl-butyric acid Intermediate 1 (125 mg, 0.38 mmol), Piperazine-1-carboxylic acid naphthalen-2-ylamide hydrochloride (110 mg, 0.38 mmol), 2,4,6-Collidine (0.10 ml, 0.76 mmol) and HATU (144 mg, 0.38 mmol) were reacted in dry DCM (10 ml) under the same conditions employed to synthesise example 1. Similar work-up and purification by preparative HPLC yielded the title compound (50 mg, 24%) as a colourless oil. LRMS: +ve ion 566 [M+H⁺, 100%]; HPLC-RT: 10.2 min; ¹H-NMR (250 MHz), δ (MeOH-d4) 8.26, 7.82 (1H, 2 bs, CHO-rotamers), 7.96-7.74 (3H, m, 3 ArH), 7.53-7.37 (4H, m, 4 ArH), 4.96 (1H, m, tert-ButylCH), 4.05-2.91 (11H, 4 m, 4 CH₂N-piperazine, HONCH₂, CH₂CHCH₂), 1.89-0.97 (20H, 2 m, 4 CH₂-cyclopentyl, CH-cyclopentyl, CH₂, C(CH₃)₃).

### EXAMPLE 9

### 4-{2S-[2R-Cyclopentylmethyl-3-(formyl-hydroxy-amino)-propionylamino]-3,3-dimethyl-butyryl}-piperazine-1-carboxylic acid benzo[1,3]dioxol-5-ylamide

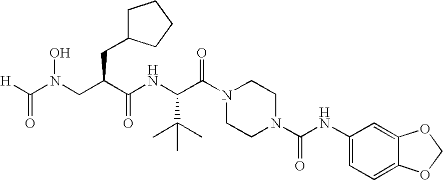

2S-[2R-Cyclopentylmethyl-3-(formyl-hydroxy-amino)-propionylamino]-3,3-dimethyl-butyric acid Intermediate 1 (200 mg, 0.62 mmol), Piperazine-1-carboxylic acid benzo[1,3]dioxol-5-ylamide hydrochloride (170 mg, 0.61 mmol), 2,4,6-Collidine (0.25 ml, 1.89 mmol) and HATU (230 mg, 0.62 mmol) were reacted in dry DCM (10 ml) under the same conditions employed to synthesise example 1. Similar work-up and purification by preparative HPLC yielded the title compound (50 mg, 15%) as a colourless oil. LRMS: +ve ion 560 [M+H⁺, 100%]; HPLC-RT: 8.9 min; ¹H-NMR (250 MHz), δ (MeOH-d4) 8.26, 7.82 (1H, 2 bs, CHO-rotamers), 6.94-6.72 (3H, 2 bs, ArH), 5.90 (2H, s, OCH₂0), 4.93 (1H, m, tert-ButylCH), 3.96-2.81 (11H, 4 m, 4 CH₂N-piperazine, HONCH₂, CH₂CHCH₂), 1.89-0.97 (20H, 2 m, 4 CH₂-cyclopentyl, CH-cyclopentyl, CH₂, C(CH₃)₃).

### EXAMPLE 10

### N{1S-[4-(Benzo[1,3]dioxol-5-ylthiocarbamoyl)-piperazine-1-carbonyl]-2,2-dimethyl-propyl}-2R-cyclopentylmethyl-3-(formyl-hydroxy-amino)-propionamide.

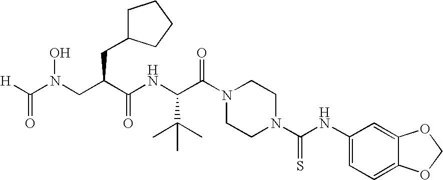

2S-[2R-Cyclopentylmethyl-3-(formyl-hydroxy-amino)-propionylamino]-3,3-dimethyl-butyric acid Intermediate 1 (252 mg, 0.77 mmol), Piperazine-1-carbothioic acid benzo[1,3]dioxol-5-ylamide hydrochloride (235 mg, 0.78 mmol), 2,4,6-Collidine (0.30 ml, 2.27 mmol) and PyAOP (399 mg, 0.77 mmol) were reacted in dry DCM (10 ml) under the same conditions employed to synthesise example 1. Similar work-up and purification by prep. HPLC yielded the title compound (140 mg, 31%) as a colourless oil. LRMS: −ve ion 574 [M−H⁺, 25%]; HPLC-RT: 9.8 min; ¹H-NMR (250 MHz), δ (CDCl₃) 10.03 (1H, bs, OH), 8.32, 7.79 (1H, 2 s, CHO-rotamers), 7.88, 6.95 (2H, 2 bs, 2 NH) 6.81-6.71 (2H, m, 2 ArH), 6.67-6.65 (1H, m, ArH), 5.92 (2H, s, OCH₂O), 4.83, 4.78 (1H, 2 d, J 9.2 and 8.5 Hz, tert-ButylCH-rotamers), 4.14-3.41 (10H, 4 m, 4 CH₂N-piperazine, HONCH₂), 2.82-2.91 (1H, m, CH₂CHCH₂), 1.73-1.04 (11H, 2 m, 4 CH₂-cyclopentyl, CH-cyclopentyl, CH₂), 1.00, 0.96 (9H, 2 s, C(CH₃)₃-rotamers).

Hydroxamates were synthesised by the following route 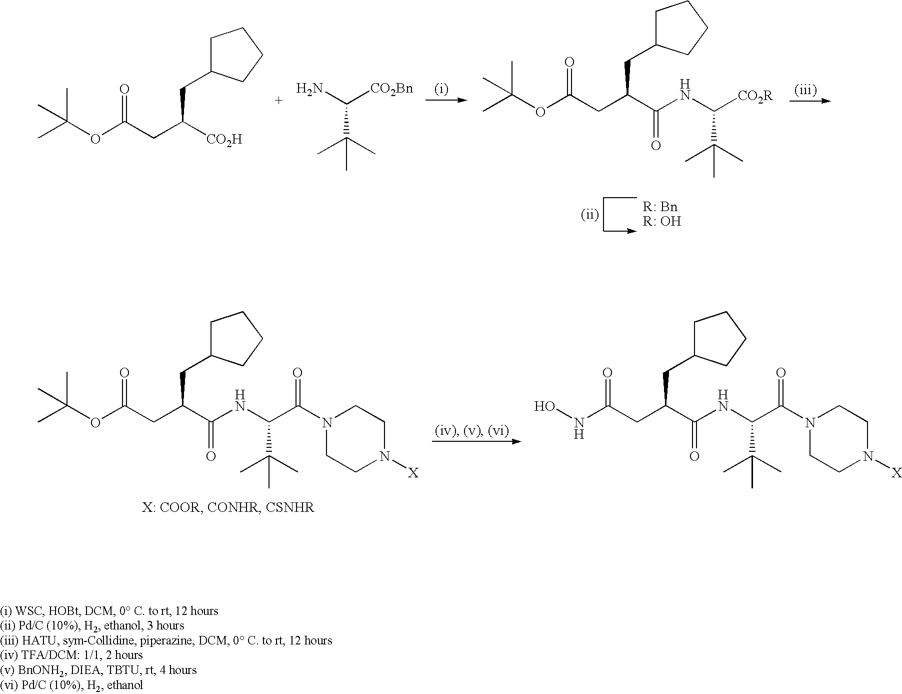
Preparation of Intermediate 3

### 2S-(3-tert-Butoxycarbonyl-2R-cyclopentylmethyl-propionylamino)-3,3-dimethyl-butyric acid benzyl ester

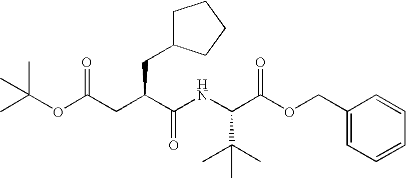

1-(3-Dimethylaminopropyl)-3-ethylcarbodiimide hydrochloride (9.0 g, 46.9 mmol), HOBt (6.0 g, 39.2 mmol) and 2S-Amino-3,3-dimethyl-butyric acid benzylester (11.2 g, 50.6 mmol) were added to a solution of 2R-Cyclopentylmethyl-succinic acid 4-teri-butylester (10.0 g 39.1 mmol) in dry DMF (100 ml) at 0° C. The ice bath was removed after 2.5 hours and the mixture was stirred for further 10 hours at room temperature. The reaction mixture was taken up in ethyl acetate (600 ml) and washed with sat. sodium bicarbonate solution (2×150 ml), water (2×150 ml) and brine (150 ml) and dried over anhydrous magnesium sulphate. Concentration and purification by silica gel flash chromatography (eluent: 4/1 hexane/ethylacetate) gave a solid material, which was recrystallised from hexane to yield the title compound (12.3 g, 68%) as colourless needles. LRMS: +ve ion 460 [M+H⁺, 20%], 482 [M+Na⁺, 50%]; ¹H-NMR (250 MHz), δ (CDCl₃) 7.36-7.30 (5H, m, ArH), 6.30 (1H, bd, J 9.4 Hz, NH), 5.15 (2H, s, CH₂Ph), 4.50 (1H, d, J 9.4 Hz,tert-ButylCh) , 2.68-2.25 (3H, 2 m, COCH₂CHCO), 1.76-0.99 (11H, m, 4 CH₂-cyclopentyl, CH-cyclopentyl, CH₂) 1.42 (9H, s, OC(CH₃)₃), 0.96 (9H, s, C(CH₃)₃)

### 2S-(3-tert-Butoxycarbonyl-2R-cyclopentylmethyl-propionylamino)-3,3-dimethyl-butyric acid

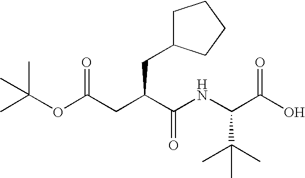

A mixture of 2S-(3-tert-Butoxycarbonyl-2R-cyclopentylmethyl-propionylamino)-3,3-dimethyl-butyric acid benzyl ester (8 g, 17.4 mmol) and Palladium-on-carbon (10%, 660 mg) in ethanol (100 ml) was stirred under an atmosphere of hydrogen for 3 hours. Filtration over celite and concentration gave an oily residue, which was taken up in ethyl acetate (500 ml) and filtered by gravitation. Removal of the solvent under reduced pressure gave the title compound (6.1 g, 95%) as a crystalline solid without the requirement for further purification. LRMS: +ve ion 370 [M+H⁺, 30%], 392 [M+Na⁺, 40%]; −ve ion 414 [M+HCO₂ ⁻, 100%], ¹H-NMR (250 MHz), δ (MeOH-d4) 7.86 (1H, bd, J 9.1 Hz, NH), 4.31 (1H, m, tert-ButylCH), 2.87 (1H, m, CH₂CHCH₂), 2.53 (1H, dd, J₁ 9.0, J₂ 16.4 Hz, 0.5 CH₂CO), 2.32 (1H, dd, J₁ 5.6, J₂ 16.4 Hz, 0.5 CH₂CO), 1.92-1.07 ((11H, m, 4 CH₂-cyclopentyl, CH-cyclopentyl, CH₂),1.43 (9H, s, OC(CH₃)₃), 1.03 (9H, s, C(CH₃)₃).

### 4-[2S-(3-tert-Butoxycarbonyl-2R-cyclopentylmethyl-propionylamino)-3,3-dimethyl-butyryl]-piperazine-1-carboxylic acid phenyl ester

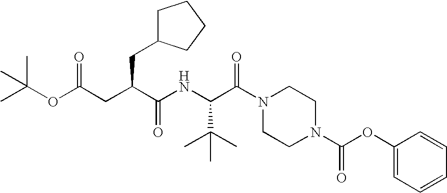

2S-(3-tert-Butoxycarbonyl-2R-cyclopentylmethyl-propionylamino)-3,3-dimethyl-butyric acid (509 mg, 1.38 mmol), Piperazine-1-carboxylic acid 4-phenyl ester hydrochloride (322 mg, 1.32 mmol), 2,4,6-Collidine (0.54 ml, 4.1 mmol) and HATU (517 mg, 1.36 mmol) were reacted in dry DCM (10 ml) under the same conditions employed to synthesise example 1. Similar work-up and purification by silica gel flash chromatography (eluent: 2/1 hexane/ethyl acetate) yielded the title compound (732 mg, 95%) as a colourless oil. LRMS: +ve ion 558 [M+H⁺, 100%], 580 [M+Na⁺, 75%]; ¹H-NMR (250 MHz), δ (CDCl₃) 7.40-7.07 (5H, m, ArH), 6.47 (1H, bd, J 9.4 Hz, NH), 4.91 (1H, d, J 9.4 Hz, tert-ButylCH), 3.99-3.49 (8H, m, 4 CH₂N-piperazine), 2.68-2.27 (3H, m, COCH₂CHCO), 1.81-1.08 (11H, m, 4 CH₂-cyclopentyl, CH-cyclopentyl, CH₂), 1.43 (9H, s, OC(CH₃)₃), 1.01 (9H, s, C(CH₃)₃).

### 4-[2S-(3-Benzyloxycarbamoyl-2R-cyclopentylmethyl-propionylamino)-3,3-dimethyl-butyryl]-piperazine-1-carboxylic acid phenyl ester (Intermediate 3)

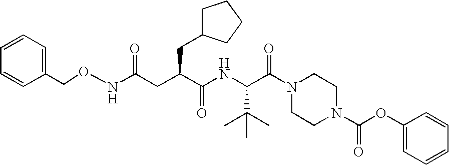

4-[2S-(3-tert-Butoxycarbonyl-2R-cyclopentylmethyl-propionylamino)-3,3-dimethyl-butyryl]-piperazine-1-carboxylic acid phenyl ester (730 mg, 1.13 mmol) was dissolved in a mixture of DCM and TFA (15 ml, 1/1) and stirred at room temperature for 2 hours. After removal of the solvent under reduced pressure the residue was once co-distilled from a mixture of methanol and water before dried in vacuo over a period of 12 hours.

The crude acid was redissolved in dry DCM (10 ml) and DIEA (0.46 ml, 2.64 mmol), O-Benzylhydroxylamine (0.16 ml, 1.31 mmol) and TBTU (420 mg, 1.31 mmol) were added. After stirring for 4 hours at room temperature the mixture was further diluted with ethyl acetate (100 ml), washed with sat. sodium hydrogencarbonate solution (25 ml), water (25 ml), brine (25ml) and dried over anhydrous magnesium sulphate. Concentration and purification of the remaining oil by silica gel flash chromatography (eluent: 4/1 toluene/acetone) yielded the title compound (Intermediate 3) (260 g, 32% over 2 steps) as a colourless oil. LRMS: +ve ion 607 [M+H⁺, 25%], 629 [M+Na⁺, 100%]; ¹H-NMR (250 MHz), δ (CDCl₃) 9.04 (1H, bs, ONH), 7.39-7.05 (10H, m, ArH), 6.70 (1H, bd, J 9.3 Hz, NH), 4.95-4.86 (3H, m, CH₂Ph, tert-ButylCH), 3.96-3.47 (8H, m, 4 CH₂N-piperazine), 2.89-1.24 (14H, 4 m, COCH₂CHCO, 4 CH₂-cyclopentyl, CH-cyclopentyl, CH₂), 1.01, 1.00 (9H, 2 s, C(CH₃)₃-rotamers).

### EXAMPLE 11

### [2S-(2R-Cyclopentylmethyl-3-hydroxycarbamoyl-propionylamino)-3,3-dimethyl-butyryl]-piperazine-1-carboxylic acid phenyl ester

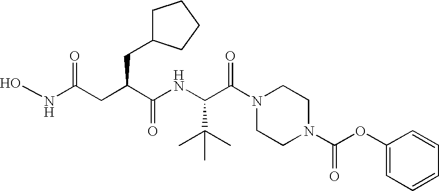

1. A compound of formula(II), or a pharmaceutical or veterinary acceptable salt, hydrate or solvate thereof 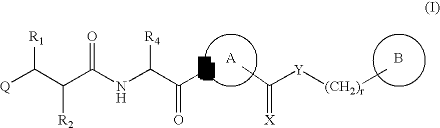
wherein:
Q represents a radical of formula —N(OH)CH(═O) or formula —C(═O)NH(OH);
R₁ represents hydrogen, methyl or trifluoromethyl or, except when Q is a radical of formula —N(OH)CH(═O), a hydroxy, halo or amino group;
R₂ represents a group R₁₀—(D)ₙ—(ALK)ₘ— wherein
R₁₀ represents hydrogen, or an optionally substituted C₁-C₆ alkyl, C₂-C₆ alkenyl, C₂-C₆ alkynyl, cycloalkyl, aryl, or heterocyclyl group and
ALK represents a straight or branched divalent C₁-C₆ alkylene, C₂-C₆ alkenylene, or C₂-C₆ alkynylene radical, and may be interrupted by one or more non-adjacent —NH—, —O—or —S— linkages,
D represents —NH—, —O— or —S—, and
m and n are independently 0 or 1;
R₄ represents the side chain of a natural or non-natural alpha amino acid; ring A represents an optionally substituted monocyclic heterocyclic ring containing from 5 to 7 ring atoms, one of which is the nitrogen atom shown, the remaining ring atoms being selected from compatible combinations of carbon, oxygen, sulfur and nitrogen; X is oxygen or sulfur;
Y is oxygen, sulfur or —NH—;
R is 0,1,2or 3; and
ring B represents an optionally substituted carbocyclic or heterocyclic ring system.

2. A compound as claimed in claim 1 wherein R is hydrogen.

3. A compound as claimed in claim 1 wherein R₂ is:
optionally substituted C₁-C8 alkyl, C₃-C₆ alkenyl, C₃-C₆ alkynyl or cycloalkyl;
phenyl (C₁-C₆ alkyl)-, phenyl (C₃-C₆ alkenyl)- or phenyl (C₃-C₆ alkynyl)- optionally substituted in the phenyl ring;
cycloalkyl (C₁-C₆ alkyl)-, cycloalkyl (C₃-C₆ alkenyl)-or cycloalkyl (C₃-C₆ alkynyl)- optionally substituted in the cycloalkyl ring;
heterocyclyl (C₁-C₆ alkyl)-, heterocyclyl (C₃-C₆ alkenyl)- or heterocyclyl (C₃-C₆ alkynyl)- optionally substituted in the heterocyclyl ring; or
CH₃ (CH₂)ₚO(CH₂)_{q}— or CH₃(CH₂)ₚS(CH₂)_{q}—, wherein p is 0, 1, 2 or 3 and q is 1, 2 or 3.

4. A compound as claimed in claim 1 wherein R₂ is methyl, ethyl, n-or iso-propyl, n- or iso-butyl, n-pentyl, iso-pentyl 3-methyl-but-1-yl, n-hexyl, n-heptyl, n-acetyl, n-octyl, methylsulfanylethyl, ethylsulfanylmethyl, 2-methoxyethyl, 2-ethoxyethyl, 2-ethoxymethyl, 3-hydroxypropyl, allyl, 3-phenylprop-3-en-1-yl, prop-2-yn-1-yl, 3-phenylprop-2-yn-1-yl, 3-(2-chlorophenyl)prop-2-yn-1-yl, but-2-yn-1-yl, cyclopentyl cyclohexyl, cyclopentylmethyl, cyclopentylethyl, cyclopentylpropyl, cyclohexylmethyl, cyclohexylethyl, cyclohexylpropyl, furan-2-ylmethyl, furan-3-methyl, tetrahydrofuran-2-ylmethyl, tetrahydrofuran-2-ylmethyl, piperidinylmethyl, phenylpropyl, 4-chlorophenylpropyl, 4-methylphenylpropyl, 4-methoxyphenylpropyl, benzyl, 4-chlorobenzyl, 4-methylbenzyl, or 4-methoxybenzyl.

5. A compound as claimed in claim 1 wherein R₂ is (C₁-C₆) alkyl-, cycloalkylmethyl-, (C₁-C₃)alkyl-S—(C₁-C₃)alkyl-, or (C₁-C₃)alkyl-O—(C₁-C₃) alkyl.

6. A compound as claimed in claim 1 wherein R₄ is:
the characterising group of a natural a amino acid or 4-methoxyphenylmethyl, in which any functional group may be protected, any amino group may be acylated and any carboxyl group present may be amidated; or
a group-[Alk]ₙR₉ where Alk is a (C₁-C₆) alkylene or (C₂-C6)alkenylene group optionally interrupted by one or more —O—, or —S—atoms or —N(R₁₂)— groups [where R₁₂ is a hydrogen atom or a (C₁-C₆) alkyl group], n is 0 or 1, and R₉ is hydrogen or an optionally substituted phenyl, aryl, heterocyclyl, cycloalkyl or cycloalkenyl group or (only when n is 1) R₉ may additionally be hydroxy, mercapto, (C₁-C₆)alkylthio, amino, halo, trifluoromethyl, nitro, —COOH, —CONH₂, —COOR^{A}, —NHCOR^{A}, —CONHR^{A}, —NHR^{A}, —NRAR^{B}, or —CONR^{A}R^{B} wherein R^{A} and R^{B} are independently a (C₁-C₆)alkyl group; or
a benzyl group substituted in the phenyl ring by a group of formula —OCH₂COR₈ where R₈ is hydroxyl, amino, (C₁-C₆) alkoxy, phenyl(C₁-C₆)alkoxy, (C₁-C₆)alkylamino, di( (C₁-C₆) alkyl)amino, phenyl(C₁-C₆)alkylamino; or a heterocyclic(C₁-C₆)alkyl group, either being unsubstituted or mono- or di-substituted in the heterocyclic ring with halo, nitro, carboxy,(C₁-C₆)alkoxy, cyano, (C₁-C₆)alkanoyl, trifluoromethyl(C₁-C₆)alkyl, hydroxy, formyl, amino,(C₁-C₆)alkylamino, di-(C₁-C₆)alkylamino, mercapto, (C₁-C₆)alkylthio, hydroxy(C₁-C₆)alkyl, mercapto(C₁-C₆)alkyl or (C₁-C₆)alkylphenylmethyl; or
a group-CRₐR_{b}R_{c} in which:
each of Rₐ, R_{b} and R_{c} is independently hydrogen, (C₁-C₆)alkyl, (C₂-C₆)alkenyl, (C₂-C₆)alkynyl, phenyl (C₁-C₆)alkyl, (C₃-C₈)cycloalkyl; or
R_{c} is hydrogen and Rₐ and R_{b} are independently phenyl or heteroaryl such as pyridyl; or
R_{c} is hydrogen, (C₁-C₆)alkyl, (C₂-C₆)alkenyl, (C₂-C₆)alkynyl, phenyl(C₁-C₆)alkyl, or ( C₃-C8)cycloalkyl, and Rₐ and R_{b} together with the carbon atom to which they are attached form a 3 to 8 membered cycloalkyl or a 5-to 6-membered heterocyclic ring; or
Rₐ, R_{b} and R_{c} together with the carbon atom to which they are attached form a tricyclic ring; or
Rₐ and R_{b} are each independently (C₁-C₆)alkyl, (C₂-C₆)alkenyl, (C₂-C₆)alkynyl, phenyl(C₁-C₆) alkyl, or a group as defined for R_{c} below other than hydrogen, or Rₐ and R_{b} together with the carbon atom to which they are attached form a cycloalkyl or heterocyclic ring, and R_{c} is hydrogen, —OH, —SH, halogen, —CN, —CO₂H, (C₁-C₄)perfluoroalkyl, —CH₂OH, —CO₂(C₁-C₆)alkyl, —O(C₁-C₆)alkyl, —O(C₂-C₆)alkenyl, —S(C₁-C₆)alkyl, —SO(C₁-C₆)alkyl, —SO₂(C₁-C₆)alkyl, —S(C₂-C₆) alkenyl, —SO(C₂-C₆) alkenyl, —SO₂(C₂-C₆) alkenyl or a group-Q-W wherein Q represents a bond or —O—, —S—, —SO— or —SO₂—and W represents a phenyl, phenylalkyl, (C₃-C₈) cycloalkyl, (C₃-C₈)cycloalkylalkyl, (C₄-C₈)cycloalkenyl, (C₄-C₈)cycloalkenylalkyl, heteroaryl or heteroarylalkyl group, which group W may optionally be substituted by one or more substituents independently selected from, hydroxyl, halogen, —CN, —CO₂H, —CO2(C₁-C₆)alkyl, —CONH₂, —CONH(C₁-C₆)alkyl, —CONH(C₁-C₆alkyl)₂, —CHO, —CH₂OH, (C₁-C₄)perfluoroalkyl, —O(C₁-C₆)alkyl, —S(C₁-C₆)alkyl, —SO(C₁-C₆)alkyl, —SO₂(C₁-C₆)alkyl, —NO₂, —NH₂, —NH(C₁-C₆)alkyl, N((C₁-C₆)alkyl)₂, —NHCO(C₁-C₆)alkyl, (C₁-C₆)alkyl, (C₂-C₆)alkenyl, (C₂-C₆)alkynyl, (C₃-C₈)cycloalkyl, (C₄-C₈)cycloalkenyl, phenyl or benzyl.

7. A compound as claimed in claim 1 wherein R₄ is methyl, ethyl, benzyl, 4-cholorobenzyl, 4-hydroxybenzyl, phenyl, cyclohexyl, cyclohexylmethyl, pyridine-3-ylmethyl, tert-butoxymethyl, naphthylmethyl, iso-butyl, sec-butyl, tert-butyl, 1-benzylthio-1-methylethyl, 1-methylthio-1-methylethyl, 1-mercapto-1-methylethyl, 1-methoxy-1-methylethyl, 1-hydroxy-1-methylethyl, 1-fluoro-1-methylethyl, hydroxymethyl, 2-hydroxethyl, 2-carboxyethyl, 2-methylcarbomoylethyl, 2-carbamoylethyl, or 4-aminobutyl.

8. A compound as claimed in claim 1 wherein R₄ is tert-butyl, iso-butyl, benzyl, isopropyl or methyl.

9. A compound as claimed in claim 1 wherein ring A is optionally substituted 1-pyrrolidinyl, piperidin-1-yl, 1-piperazinyl, hexahydro-1-pyridazinyl, morpolin-4-yl, tetrahydro-1,4-thiazin4-yl, tetrahydro-1,4-thiazin-4-yl 1-oxide, tetrahydro-1,4-thiazin-4-yl 1,1-dioxide, hexahydroazipino, thiomorpholino, diazepino, thiazolidinyl or octahydroazochino.

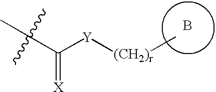

10. A compound as claimed in claim 1 wherein ring A is piperidin-1-yl or 1-piperazin-4-yl.

11. A compound as claimed in claim 1 wherein the grouping 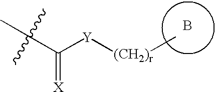
present in compounds(I) is attached to a ring carbon atom or a second ring nitrogen atom of ring A.

12. A compound as claimed in claim 1 wherein r is 0 or 1.

13. A compound as claimed in claim 1 wherein ring B is optionally substituted phenyl, 2-, 3- or 4-pyridyl, 9H-fluoren-9-yl, naphthyl, or 4-benzo[1,3]dioxol-5-yl.

14. A compound as claimed in claim 1 wherein in the grouping 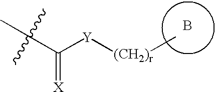
present in compounds(I), X is oxygen or sulphur when Y is —NH—, or both X and Y are oxygen.

15. (canceled)

16. (canceled)

17. A method for the treatment of microbial infections in humans and nonhuman mammals, which comprises administering to a subject suffering such infection an antimicrobially effective dose of a compound as claimed in claim 1.

18. An antimicrobial composition comprising a compound as claimed in claim 1 together with a pharmaceutical acceptable carrier.

19. A compound as claimed in claim 5 wherein R₂ is n-propyl, n-butyl, n-pentyl, cyclopentylmethyl, cyclopentylethyl, cyclohexylmethyl or cyclohexylethyl

20. A compound as claimed in claim 2 wherein R₂ is:
optionally substituted C₁-C8 alkyl, C₃-C₆ alkenyl, C₃-C₆ alkynyl or cycloalkyl;
phenyl (C₁-C₆ alkyl)-, phenyl (C₃-C₆ alkenyl)- or phenyl (C₃-C₆ alkynyl)- optionally substituted in the phenyl ring;
cycloalkyl (C₁-C₆ alkyl)-, cycloalkyl (C₃-C₆ alkenyl)- or cycloalkyl (C₃-C₆ alkynyl)- optionally substituted in the cycloalkyl ring;
heterocyclyl (C₁-C₆ alkyl)-, heterocyclyl (C₃-C₆ alkenyl)- or heterocyclyl (C₃-C₆ alkynyl)-optionally substituted in the heterocyclyl ring; or
CH₃ (CH₂)ₚO(CH₂)_{q}13 or CH₃(CH₂)ₚS(CH₂)_{q}—, wherein p is 0, 1, 2 or 3 and q is 1, 2 or 3.

21. A compound as claimed in claim 2 wherein R₂ is methyl, ethyl, n-or iso-propyl, n-or iso-butyl, n-pentyl, iso-pentyl 3- methyl-but-1-yl, n-hexyl, n-heptyl, n-acetyl, n-octyl, methylsulfanylethyl, ethylsulfanylmethyl, 2-methoxyethyl, 2-ethoxyethyl, 2-ethoxymethyl, 3-hydroxypropyl, allyl,3-phenylprop-3-en-1-yl, prop-2-yn-1-yl, 3- phenylprop-2-yn-1-yl, 3-(2-chlorophenyl)prop-2-yn-1-yl, but-2-yn-1-yl, cyclopentyl, cyclohexyl, cyclopentylmethyl, cyclopentylethyl, cyclopentylpropyl, cyclohexylmethyl, cyclohexylethyl, cyclohexylpropyl, furan-2-ylmethyl, furan-3-methyl, tetrahydrofuran-2-ylmethyl, tetrahydrofuran-2-ylmethyl, piperidinylmethyl, phenylpropyl, 4-chlorophenylpropyl, 4-methylphenylpropyl, 4-methoxyphenylpropyl, benzyl, 4-chlorobenzyl, 4-methylbenzyl, or 4-methoxybenzyl.

22. A compound as claimed in claim 2 wherein R₂ is (C₁-C₆) alkyl-, cycloalkylmethyl-, (C₁-C₃)alkyl-S—(C₁-C₃)alkyl-, or (C₁-C₃)alkyl-O—(C₁-C₃) alkyl-.
